(19) United States
(12) Patent Application Publication
(10) Pub. No.: US 2004/0142392 A1
(43) Pub. Date: Jul. 22, 2004

(54) Method for determining analyte molecules in a sample

(76) Inventors: Markus Templin, Tuebingen (DE); Thomas Joos, Tuebingen (DE); Stefan Lampel, Germering (DE); Volker Schlueter, Muenchen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/680,371

(22) Filed: Oct. 7, 2003

Related U.S. Application Data
(63) Continuation of application No. PCT/EP02/03730, filed on Apr. 4, 2002.

(30) Foreign Application Priority Data
Apr. 10, 2001 (EP) 01 108 907.5

(51) Int. Cl. G01N 33/53

(52) U.S. Cl. 435/7.9

(57) ABSTRACT

This invention relates to a method for determining the concentration/amount of analyte molecules in the sample. The method has at least the following steps: a) adding to the sample a known concentration/amount of competitor or analogue molecules of the analyte, which are distinct from the analyte molecules, b) reacting/contacting the sample of step a) with capture molecules for the analyte and competitor molecules, c) determining a measure for the ratio R of analyte and competitor molecules that are bound to the capture molecules, d)determining the concentration/amount of the analyte molecules from the ratio R of step c) and the known concentration/amount of competitor molecules in the sample of step a).

### CROSS-REFERENCES TO RELATED APPLICATIONS

This application is a continuation of co-pending international patent application PCT/EP02/03730 filed on Apr. 4, 2002 and designating the US, which was published under PCT Art. 21 (2) in English, and claims priority of European patent application EP 01 108 907.5 filed Apr. 10, 2001, which is incorporated herein by reference.

### BACKGROUND OF THE INVENTION

1. Field of the Invention

The invention relates to the field of chemical analysis, relating particularly to the field of diagnostic analysis and most particularly to protein detection and analysis, proteomics and clinical screening procedures.

Especially, the application relates to a method for determining, preferably quantifying analyte molecules in a sample and to a kit that comprises reagents suitable in performing the method.

2. Description of the Related Art

One example of a generic detection method is a so-called Sandwich-Immune-Assay that might be performed on a microarray. Analytical procedures using microarrays are known in the art. Capture molecules or antibodies with a known affinity to an analyte are immobilized and the amount of analyte bound to the capture molecules is measured. The binding of the capture antibody is facilitated by specific modifications on the surface of the support. After binding of the capture antibody an incubation with individual samples is performed followed by reaction with a labelled probe, that can be, e.g., a second sample-directed antibody suitable for completing the antibody-sample “sandwich”.

The detection antibody is labelled with a radio- or fluorescence label or, in an enzyme-linked immunosorbent assay (“ELISA”), is coupled to an enzyme in order to catalyze a chemical reaction generating a detectable precipitate as product. In each case, the primary data are measured and the amount of captured analyte can be calculated using defined concentration of the analyte in an unknown sample.

Microarray based procedures using this principle have been described in the prior art. One method relies on the assumption that the amount of captured analyte does not change the concentration of the analyte in the sample significantly and therefore the mass action law can be used to calculate the concentration in the unknown sample (ambient analyte assay systems). However, in many applications this assumption fails to lead to reliable results.

Competitive Assays for the quantification of an analyte can also be used to determine unknown concentrations in a sample. Commonly used assays are the competitive radioactive immunosorbent assay (RIA) procedures and the competitive enzyme linked immunosorbent assay (ELISA). In both cases a labelled analyte to be measured is included in the reaction mixture. During sample incubation a competitive reaction between labelled standard and sample protein creates a signal which is used to determine the analyte concentration via a series of standards.

This prior art is accompanied by several severe disadvantages, namely:

to calibrate the assay several standards have to be included to allow a reliable quality control; multiple standards are needed;

quality of the obtained data is highly dependent on the quality of the spot and methods to correct or to minimize spot to spot variation have to be introduced to obtain re-liable results;

the described microarray based systems have to use an optimal working concentration that is determined by the binding constants (capture molecule and analyte) and the amount of the capture molecules used. Therefore, the concentration of the analyte in the assay has to be adjusted to an optimal value to allow the method to work reliably. This points to a principle disadvantage of the described assay systems in general. When applying the method for a simultaneous detection of multiple analytes in one assay, it is difficult to adjust the concentration of each analyte to be quantified to its optimal value. This presents a major problem since the concentration of different analytes to be quantified may vary by several orders of magnitude. The problem is a major obstacle when using the ambient analyte assay systems as described by Ekins in U.S. Pat. No. 5,807,755, because measurements performed at non-optimal conditions suffer from a high intrinsic experimental error.

U.S. Pat. No. 5,834,319 discloses a method for determining the concentration of an analyte by binding it to a surface in non-saturating quantities. A back-titration is then made with a labelled analogue of the analyte i.e. a first developing reagent, followed by reaction with a second developing reagent, which is also labelled. Not only is a labelled analyte analogue required in this method, but this back titration requires that the labelled analogue be added after the ambient assay to saturate binding sites which were not occupied by the analyte in the sample, thereby extending the procedure and entailing additional washing steps in which bound analyte dissociates from the capture molecules.

WO 00/63701 discloses microarrays of polypeptides on a solid support for multiplexed detection and quantitation of ligands. The ligands are labelled directly and detected without a detection reagent being used. The method is therefore inflexible in terms of responsiveness and range of analyte concentrations over which it may be usefully employed.

### SUMMARY OF THE INVENTION

In view of the above, it is an object of the present invention to avoid and/or circumvent the drawbacks of the prior art and to provide a reliable, easy to perform method for determining the amount or concentration of an analyte in a sample.

This object is achieved with a method as detailed in independent claims 1 or 17 and a kit as in independent claims 13 or 24. Preferred embodiments of the invention are subject of the dependent claims.

According to one object, the method comprises the steps of:

a) adding to the sample a known concentration/amount of competitor or analogue molecules of the analyte, said competitor/analogue molecules being distinct from said analyte molecules,

b) reacting/contacting the sample of step a) with capture molecules for the analyte and competitor molecules,

c) determining a measure for the ratio R of analyte and competitor molecules that are bound to the capture molecules,

d) determining the concentration/amount of the analyte molecules from the ratio R of step c) and the known concentration/amount of competitor molecules in the sample of step a).

According to another object, the method comprises the steps of:

a) adding to the sample a known concentration/amount of competitor/analogue molecules of the analyte, said competitor/analogue molecules being distinct from said analyte molecules,

b) reacting/contacting the sample of step a) with capture molecules for the analyte and competitor/analogue molecules,

c) determining the amount of competitor/analogue molecules bound to the capture molecules using a detection reagent, preferably a labeled or (enzyme) conjugated ligand, or labeled competitor/analogue molecules, and determining the total amount of analyte and competitor/analogue molecules bound to the capture molecules using a detection reagent, preferably a labeled or (enzyme) conjugated ligand, and calculating therefrom a measure for the ratio R of analyte and competitor/analogue molecules that are bound to the capture molecules,

d) determining the concentration/amount of the analyte molecules from the ratio R of step c) and the known concentration/amount of competitor/analogue molecules in the sample of step a).

The invention can be performed in solution, suspension (beads, virtual array) or in an array format. An important feature of the invention is that the competitor/analogue molecules are distinct from the analyte molecules, i.e. can be distinguished therefrom. To this end the analyte and/or competitor/analogue molecules can be labelled or conjugated, e.g., to an enzyme. In case the analyte molecules are already labelled, unlabelled competitors can be used, a detection reagent being provided that allows determination of the sum of analyte and competitor/analogue molecules bound to the capture molecules.

Another option is to use at least two differently labelled antibodies that bind either the analyte or the competitor.

The binding constant between analyte and capture molecules can be equal to or different from the binding constant between competitor and capture molecules. From their experiments, inventors have recognized that the relationship between the concentrations/amounts of analyte and competitor molecules can either be calculated based upon the law of mass action or taken from a calibration curve.

The inventive method has several advantages:

The need for standards is reduced. It is sufficient to know the exact amount of competing, preferably labelled analyte analogue or competitor present in the assay to get valid information about the analyte concentration to be determined. It is not necessary to know the concentration of the capture molecules.

The invention allows to analyze a large number of analytes simultaneously, even when their concentrations are different. The assay can be performed under saturating conditions were conventional systems don't work any more, since the amount of added standard (competitor), which is for generating the primary signal, can be adjusted individually. The method as such provides internal standards for quantification, leading to reliable and robust assay procedures. Inter-assay and intra-assay variations are efficiently eliminated by internal standardization. Assay is not dependent on the affinity constants of the capture molecules. Characterized capture and detection reagents (e.g., ELISA reagents) can be employed with minimal operating expense.

A new combination of competitive and non-competitive assays in one reaction is provided. The method helps to circumvent the major disadvantages of prior art microarray based detection systems. This new combination allows the parallel detection of multiple parameters and therefore allows to determine the concentration of a high number of analytes at the same time.

Conventional assay systems using protein microarrays depend on the optimal amount of capture molecule immobilized and on a fitting concentration of the analyte to be quantified. The “ambient analyte assay system” as described in U.S. Pat. No. 5,834,319 is working accurately only when a small (<5%) proportion of the analyte gets bound to the capture molecule. This is a major drawback when planning to quantify different analytes that are present in differing concentrations (differences of several orders of magnitude) in one sample using only one reaction.

The invention uses in a preferred embodiment the addition of a labelled analogue of the analyte to be determined. A known amount of the labelled analogue of the analyte is added to the sample and on immobilized capture molecules the labelled analogue and the unlabelled analyte get captured. The ratio R of the two substances captured on a micro-spot presents a value that can be used to calculate the unknown amount of analyte. To determine a measure for the ratio R, the competing labelled analyte analogue is detected by its introduced label. The analyte or the sum of analyte and analyte analogue present in the sample is in a preferred embodiment detected by a specific detection reagent like a secondary antibody.

This detection reagent can be specific for both the analyte to be quantified and the labelled analogue of the analyte. The obtained measurements provide information on (i) the amount of competitor captured and (ii) on the sum of competitor and analyte captured. These data are also sufficient to calculate the amount of unknown analyte.

This detection reagent can also be specific for the analyte to be quantified and therefore not be able to recognize the labelled analogue of the analyte. An assay of this type corresponds to a competitive system where competitor can be quantified and a simultaneous detection of analyte that is achieved by a different system. Both reactions occur at the same time in the same assay and therefore can be used to quantify the analyte by two different methods at the same time.

Both methods are working independently of the actual concentration of the analyte. The assays can be performed when only a fraction of the analyte is captured by the immobilized capturing reagent sample (ambient analyte assay system, no significant change of the concentration in the liquid phase) or when a significant proportion of analyte is removed from solution by the capture molecules. The method can be performed under conditions where the capture molecules are saturated by bound analyte (conventional systems don't work), since the ratio R of the competing labelled analyte analogue and the analyte is used for the concentration determination.

Assay variations and spot irregularities do not influence the determined ratios significantly. The need for controls is greatly reduced.

Applications of the new method are:

Screening: Diagnostic screening procedures (concentration determination of immunoglobulins, interleukins, etc.), screening systems for the pharmaceutical industry.

Profiling of biomolecules: Quantification of high abundant and low abundant proteins or peptides; Quantification of high affinity and low affinity binding proteins or peptides, sugars, nucleic acids and the like.

### BRIEF DESCRIPTION OF THE DRAWINGS

FIG. 1 shows a sketch of a plot of the ratio R of Cy5 and Cy3 signals obtained from quantification of fluorescent signals; and

FIG. 2 shows a sketch of data obtained comparable to those of FIG. 1.

### DETAILED DESCRIPTION OF THE PREFERRED EMBODIMENT

### EXAMPLE

### Quantification of IgG3

a. Reagents

1. Cy3 mAb Labelling Kit (PA33001) from Amersham-Pharmacia.

2. Filter spin units “Microcon 10” from Amicon.

3. Tween-20, TRIS-base, Thimerosal from Sigma Chemical Co. (St. Louis, Mo., USA); bovine serum albumin Fraction V (BSA) from PAA Laboratories; PBS from Eurobio; low fat dried milk from Heirle; sodium carbonate decahydrate from Fluka.

4. Aldehyde activated glass slides (silylated slidesCSS) from Telechem.

5. Rat anti mouse IgG3 monoclonal capture antibodies (Clone R2-38) and rat anti mouse IgG3 monoclonal Biotin-conjugated developing antibodies (Clone R40-82), Mouse IgG3 standard (Clone A112-3) from Pharmingen.

6. Cy5-conjugated Streptavidin from Amersham Pharmacia.

b. Creation of Microarrays

Rat anti mouse IgG3 monoclonal capture antibodies (conc. 500 μg/ml in PBS) were arrayed using a GMS 417 micro arrayer, three dots (equivalent to 150 pl) were deposited to a given spot. Arrays were created on aldehyde activated slides (CSS slides, Telechem). After printing the slides were incubated for 12 h in a wet chamber and stored dry in the dark.

c. Labelling and Purification of Proteins with Dyes Fluorescent

1. 100 μg IgG3 standard (A 112-3) at a concentration of 1 mg/ml was used for the labelling reaction. Coupling with Cy3 was performed as described by the manufacturer.

2. To terminate the reaction IM TRIS-HCl pH 8.0 was added to a final concentration of 100 mM and incubated in the dark for 30 min.

3. The labelling mix was transferred to a “Microcon 10” concentration unit, adjusted to 400 μl with PBS and centrifuged for 25 min at 12,000 g. PBS was added to the protein solution to give a volume of 400 μl and the concentration procedure was repeated twice to wash away free label.

The purified Cy3-labeled IgG3 was brought to a volume of 200 μl by adding PBS containing 0.05% Thimerosal.

d. Combined Competitive/Non-Competitive Assay for the Quantification of IgG3.

1. Slides were blocked in PBS containing 1.5% BSA and 5% low fat dried milk powder, 0.1% Tween-20 for 1 h and washed briefly in PBS containing 0.1% Tween-20.

2. Serum samples, i. e. unlabelled IgG3 serving as analyte, were diluted (1:40, 1:60, 1:70, 1:80, 1:90, 1:100, 1:160) in 25 μl pas containing 1.5% BSA, 2.5% low fat dried milk and Cy3-labeled IgG3 was added as competitor (1:1,000). The arrays were incubated with the sample containing analyte and competitor for 40 min at RT. The relative concentration ratios between competitor and analyte were thus: 1:25, 1:16.67, 1:14.29, 1:12.5, 1:11.1, 1:10, and 1:6.25, respectively.

3. The arrays were washed five times in PBS, 0.1% Tween-20 for 1 min at RT.

4. Biotin-conjugated developing antibody was diluted in PBS containing 1.5% BSA and 2.5% low fat dried milk powder, 0.1% Tween-20 1:800 (0.625 μg) and the array incubated therewith for 40 min at RT and washed as described under 3.

5. Streptavidin-Cy5 was diluted 1/200 in PBS containing 1.5% BSA and 2.5% low fat dried milk powder, 0.1% Tween-20. The assays were incubated therewith for 20 min at RT and washed as described under 3.

6. The slides were rinsed once with water, once with distilled water and dried in a stream of nitrogen. Pictures of the fluorescent signals of Cy3 and Cy5 dyes were taken using a GMS418 array scanner. The Cy5 signal is a measure for the concentration/amount of the sum of analyte and competitor bound to the capture molecule. The Cy3 signal is a measure for the concentration/amount of competitor bound to the capture molecules.

Quantification of the obtained fluorescence signals was done with the ImaGene 4.0 software. The ratio R of the Cy5 and the Cy3 signals was calculated and plotted against the relative concentration (1:160 dilution was set to 1)of the analyte.

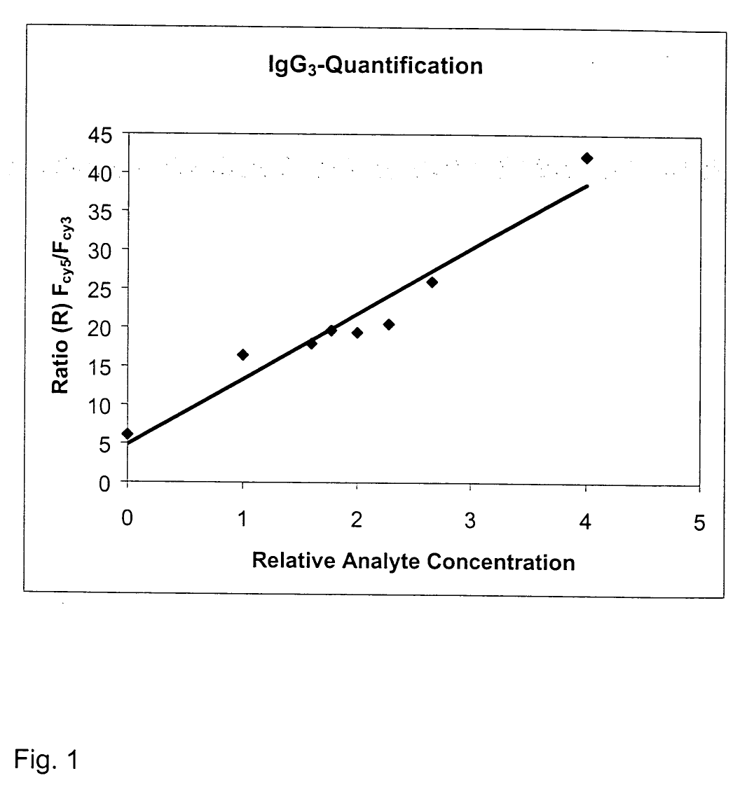

FIG. 1 shows a sketch of this plot.

### EXAMPLE 2

### Quantification of Recombinant IgG

Essentially the same experimental procedures as described above were applied to a different type of sample (recombinant IgG).

In contrast to the first example the concentration of analyte in this experiment was varied from 0.3 μg/ml to 200 μg/ml (dilution: 3, 3, 27, 81, 243, 729). The Cy3-labelled competitor was diluted 1:1,000.

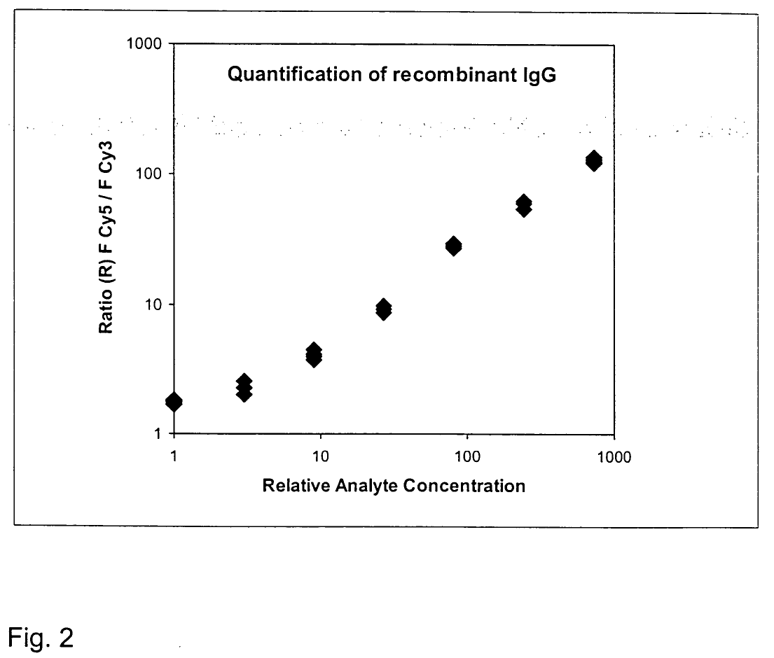

FIG. 2 shows a sketch of the obtained data.

Both FIGS. 1 and 2 show that once a calibration curve or numerical relationship between the determined ratio R and the relative analyte concentration/amount has been obtained, unknown concentrations/amounts of analyte molecules can be determined from the ratio R and the known concentration/amount of competitor/analogue molecules. The concentration/amount can either be in spread narrow range or a wide range as in FIG. 2., where the relative analyte concentration covers a range between 1 to 1,000.

In case the analyte molecules are labelled with a dye A and the competitor molecules with dye B, the ratio R would be calculated as R=(A+B)/B. However, plotting a ratio R*=A/B=R−1, against the relative or absolute analyte concentration would also provide a suitable calibration curve.

What is claimed is:

1. A method for determining the concentration/amount of analyte molecules in a sample, comprising the steps of:
a) adding to the sample a known concentration/amount of competitor or analogue molecules of the analyte, said competitor/analogue molecules being distinct from said analyte molecules,
b) reacting/contacting the sample of step a) with capture molecules for the analyte and competitor molecules,
c) determining a measure for the ratio R of analyte and competitor molecules that are bound to the capture molecules,
d) determining the concentration/amount of the analyte molecules from the ratio R of step c) and the known concentration/amount of competitor molecules in the sample of step a).

2. The method of claim 1, wherein in step c) the amount of competitor molecules bound to the capture molecules is determined using a detection reagent.

3. The method of claim 2, wherein said detection reagent is a labelled or enzyme-conjugated ligand.

4. The method of claim 1, wherein in step c) the amount of analyte molecules bound to the capture molecules is determined using a detection reagent.

5. The method of claim 4, wherein said detection reagent is a labelled or enzyme-conjugated ligand.

6. The method of claim 4, wherein in step b) analyte and competitor molecules not reacted with/bound to the capture molecules are removed.

7. The method of claim 6, wherein said analyte and competitor molecules not reacted with/bound to the capture molecules are removed by washing steps.

8. The method of claim 4, wherein the method is performed on an array of immobilized capture molecules.

9. The method of claim 1, wherein in step c) the total amount of analyte and competitor molecules bound to the capture molecules is determined using a detection reagent.

10. The method of claim 9, wherein said detection reagent is a labelled or enzyme-conjugated ligand.

11. The method of claim 1, wherein in step c) the amount of competitor molecules bound to the capture molecules is determined distinct but parallel the to determination of analyte molecules bound to the capture molecules.

12. The method of claim 1, wherein in step b) analyte and competitor molecules not reacted with/bound to the capture molecules are removed.

13. The method of claim 12, wherein said analyte and competitor molecules not reacted with/bound to the capture molecules are removed by washing steps.

14. The method of claim 1, wherein the method is performed on an array of immobilized capture molecules.

15. The method of claim 1, wherein the method is performed in a suspension capture of beads having attached there to molecules.

16. The method of claim 1, wherein the competitor molecules are labelled.

17. The method of claim 16, wherein the competitor molecules are labelled with a fluorescent dye.

18. The method of claim 1, wherein the analyte molecules are labelled.

19. The method of claim 18, wherein the analyte molecules are labelled with a fluorescent dye

20. A kit for performing the method of claim 1, comprising capture molecules.

21. The kit of claim 20, further comprising detection reagents for analyte and/or for competitor molecules.

22. The kit of claim 20, further comprising competitor molecules.

23. The kit of claim 20, wherein the capture molecules are arranged in an array.

24. The kit of claim 23, wherein said array is a microarray.

25. The kit of claim 20, wherein the capture molecules are attached to beads.

26. A method for determining the concentration/amount of analyte molecules in a sample, comprising the steps of:
a) adding to the sample a known concentration/amount of competitor/analogue molecules of the analyte, said competitor/analogue molecules being distinct from said analyte molecules,
b) reacting/contacting the sample of a) with capture step molecules for the analyte and competitor/analogue molecules,
c) determining the amount of competitor/analogue molecules bound to the capture molecules using a detection reagent or labeled competitor/analogue molecules, and determining the total amount of analyte and competitor/analogue molecules bound to the capture molecules using a detection reagent, and calculating therefrom a measure for the ratio R of analyte and competitor/analogue molecules that are bound to the capture molecules,
d) determining the concentration/amount of the analyte molecules from the ratio R of step c) and the known concentration/amount of competitor/analogue molecules in the sample of step a).

27. The method of claim 26, wherein said detection reagent is a labeled or enzyme-conjugated ligand.

28. The method of claim 26, wherein in step c) the amount of competitor/analogue molecules bound to the capture molecules is determined distinct but parallel to the determination of the total amount of competitor/analogue and analyte molecules bound to the capture molecules.

29. The method of claim 26, wherein in step b) analyte and competitor/analogue molecules not reacted with/bound to the capture molecules are removed.

30. The method of claim 29, wherein in step b) analyte and competitor/analogue molecules not reacted with/bound to the capture molecules are removed by washing steps

31. The method of claim 28, wherein in step b) analyte and competitor/analogue molecules not reacted with/bound to the capture molecules are removed.

32. The method of claim 31, wherein in step b) analyte and competitor/analogue molecules not reacted with/bound to the capture molecules are removed by washing steps.

33. The method of claim 26, wherein the method is performed on an array of immobilized capture molecules.

34. The method of claim 26, wherein the method is performed in a suspension of beads having attached thereto capture molecules.

35. The method of claim 26, wherein the competitor/analogue molecules are labeled with a fluorescent dye.

36. A kit for performing the method of claim 26, comprising capture molecules, competitor/analogue molecules, and detection reagents for analyte and for competitor/analogue molecules.

37. The kit of claim 36, wherein the capture molecules are arranged in an array.

38. The kit of claim 37, wherein said array is a microarray.

39. The kit of claim 36, wherein the capture molecules are attached to beads.

40. The kit of claim 39, wherein said beads have attached thereto different species of capture molecules for determining different species of analyte molecules.
